(19) United States
(12) Patent Application Publication
(10) Pub. No.: US 2024/0023926 A1
(43) Pub. Date: Jan. 25, 2024

(54) MODEL-STABILIZED DIAPHRAGM ULTRASONOGRAPHY MONITORING

(71) Applicant: KONINKLIJKE PHILIPS N.V., EINDHOVEN (NL)

(72) Inventors: Rafael Wiemker, Hamburg (DE); Roberto Buizza, Eindhoven (NL); Jaap Roger Haartsen, Eindhoven (NL); Cornelis Petrus Hendriks, Eindhoven (NL); Thomas Koehler, Hamburg (DE); Michael Polkey, London (GB); Joerg Sabczynski, Hamburg (DE); Nataly Wieberneit, Hamburg (DE)

(21) Appl. No.: 18/207,263

(22) Filed: Jun. 8, 2023

Related U.S. Application Data
(60) Provisional application No. 63/389,377, filed on Jul. 15, 2022.

(51) Int. Cl. A61B 8/08; A61B 8/00; G06T 7/33; G06T 7/00

(52) U.S. Cl. CPC A61B 8/0875; A61B 8/4483; G06T 7/344; G06T 7/0014; G06T 2207/10081; G06T 2207/20084

(57) ABSTRACT

A diaphragm measurement device includes a non-transitory storage medium storing a patient-specific registration model for referencing ultrasound imaging data to a reference frame. At least one electronic processor is programmed to perform a diaphragm measurement method including receiving ultrasound imaging data of a diaphragm of a patient during inspiration and expiration while the patient undergoes mechanical ventilation therapy with a mechanical ventilator; calculating a diaphragm thickness metric based on the received ultrasound imaging data of the diaphragm of the patient referenced to the reference frame using the patient-specific registration model; and displaying, on a display device, a representation of the calculated diaphragm thickness metric.

### CROSS-REFERENCE TO RELATED APPLICATIONS

This patent application claims the priority benefit under 35 U.S.C. § 119(e) of U.S. Provisional Application No. 63/389,377, filed on Jul. 15, 2022, the contents of which are herein incorporated by reference.

The following relates generally to the respiratory therapy arts, mechanical ventilation arts, mechanical ventilation monitoring arts, ventilator induced lung injury (VILI) arts, mechanical ventilation weaning arts, and related arts.

### BACKGROUND

A diaphragm thickening fraction (TFdi or TFDI) as measured by ultrasound (US) is widely recognized as a metric to evaluate diaphragm function to optimize ventilator support and weaning. The TFdi is defined as the percentage increase in diaphragm thickness relative to end-expiratory thickness during tidal breathing. The TFdi depends on diaphragmatic activity and reflects the diaphragm work of breathing (WoB) (i.e., the respiratory effort) (see, e.g., Vivier E, Mekontso Dessap A, Dimassi S, et al. Diaphragm ultrasonography to estimate the work of breathing during non-invasive ventilation. Intensive Care Med 2012; 38: 796-803; Goligher E C, Fan E, Herridge M S, et al. Evolution of diaphragm thickness during mechanical ventilation. Impact of inspiratory effort. Am J Respir Crit Care Med 2015; 192: 1080-1088).

Diaphragm thickness and strain can be assessed at the zone of apposition (ZA) during inspiration and expiration, using a linear high frequency ultrasound transducer typically operating at around 10-15 MHz. The zone of apposition is the chest wall area where the lower rib cage reaches the abdominal contents. The ultrasound probe is positioned between the antero-axillary and mid-axillary lines, perpendicular to the chest wall. With ultrasonic B-mode, the hemi-diaphragm is identified beneath the intercostal muscles as a hypo-echogenic layer of muscle tissue located between two hyper-echogenic lines (the pleural line and the peritoneal line) (see, e.g., Fayssoil A, Behin A, Ogna A, et al. Diaphragm: Pathophysiology and Ultrasound Imaging in Neuromuscular Disorders. J Neuromuscul Dis. 2018). Diaphragmatic thickening is assessed by the thickening fraction (TFdi), calculated as the percentage inspiratory increase in the diaphragm thickness relative to end-expiratory thickness (Tₑₑ) during tidal breathing, i.e., according to Equation 1:

$$TFdi = \frac{{T}_{ei} - {T}_{ee}}{{T}_{ee}} * 100 \% \qquad ( 1 )$$

with Tₑₗ as the end-inspiratory thickness.

Some studies have evaluated the correlation between TFdi and respiratory effort (see, e.g., M. Umbrello et al. Diaphragm ultrasound as indicator of respiratory effort in critically ill patients undergoing assisted mechanical ventilation: a pilot clinical study. Crit Care. 2015). This study found a correlation coefficient of R=0.8 between TFdi and oesophageal pressure-time product and R=0.7 between TF_{di} and diaphragmatic pressure-time product. In another study (see, e.g., E. Oppersma et al. Functional assessment of the diaphragm by speckle tracking ultrasound during inspiratory loading. J Appl Phys. 2017), at the zone of apposition the diaphragm strain can similarly be measured in real-time. For example, in this study, the functional assessment of the diaphragm by speckle tracking ultrasound during inspiratory loading was analyzed. The technique of speckle tracking ultrasound allows for the detection and tracking of diaphragmatic strain over time by analyzing acoustic markers called speckles. These speckles are formed by interference of ultrasound waves that are scattered from physical structures of a size comparable to the wavelength of the ultrasound waves. Both diaphragm strain and diaphragm strain rate were highly correlated to transdiaphragmatic pressure Pdi (strain r²=0.72; strain rate r²=0.80) and EAdi (strain r²=strain rate r²=0.66).

The use of ultrasound to evaluate the respiratory muscle function (especially the diaphragm) is relatively new and remains infrequent due to the difficulty in obtaining adequate measurements (see, e.g., Aarab Y, Jaber S, De Jong A, Diaphragm Ultrasonography in ICU: Why, How, and When To Use It? ICU Management & Practice, Volume 21—Issue 3, 2021; Tuinman, P. R., Jonkman, A. H., Dres, M. et al. Respiratory muscle ultrasonography: methodology, basic and advanced principles and clinical applications in ICU and ED patients—a narrative review. Intensive Care Med 46, 594-605 (2020)). Possible confounders reducing the reproducibility and accuracy of, for example, daily bed-side TFdi measurements are varying conditions between the daily measurements, such as a location of the US-probe at each measurement on the patient, an attitude (angulation) of the probe, a phase point in the patient's respiratory cycle, a manually exerted skin pressure of the probe (which can alter position of the probe respective to the diaphragm), user-chosen ultrasound settings, the location across the diaphragm at which TFdi is evaluated (since the thickening fraction can vary over the extent of the diaphragm muscle), and so forth.

The following discloses certain improvements to overcome these problems and others.

### SUMMARY

In one aspect, a diaphragm measurement device includes a non-transitory storage medium storing a patient-specific registration model for referencing ultrasound imaging data to a reference frame. At least one electronic processor is programmed to perform a diaphragm measurement method including receiving ultrasound imaging data of a diaphragm of a patient during inspiration and expiration while the patient undergoes mechanical ventilation therapy with a mechanical ventilator; calculating a diaphragm thickness metric based on the received ultrasound imaging data of the diaphragm of the patient referenced to the reference frame using the patient-specific registration model; and displaying, on a display device, a representation of the calculated diaphragm thickness metric.

In another aspect, a diaphragm measurement method includes, with at least one electronic controller, receiving ultrasound imaging data of a diaphragm of a patient during inspiration and expiration while the patient undergoes mechanical ventilation therapy with a mechanical ventilator; calculating a diaphragm thickness metric based on the received ultrasound imaging data of the diaphragm of the patient referenced to a reference frame using a patient-specific registration model; and displaying, on a display device, a representation of the calculated diaphragm thickness metric.

One advantage resides in providing accurate and reproducible monitoring of the diaphragm thickness of a patient receiving mechanical ventilation therapy.

Another advantage resides in providing feedback control of a mechanical ventilation system based on feedback from an ultrasound system that monitors a diaphragm muscle response of a patient.

Another advantage resides in automatically adjusting settings of a mechanical ventilator to help wean patients off mechanical ventilation therapy.

Another advantage resides in providing mechanical ventilation therapy without the use of invasive catheters or dedicated ventilation maneuvers for measuring respiratory mechanics.

Another advantage resides in using a detected thickening fraction of the diaphragm to wean a patient off of mechanical ventilation therapy.

Another advantage resides in a controlled muscle training and response measurement, thereby providing a “diaphragm protective” method.

Another advantage resides in using ultrasound to non-invasively measure a diaphragm response.

Another advantage resides in using ultrasound to measure a diaphragm response independent of patient effort.

Another advantage resides in providing a mechanical ventilation monitoring workflow for clinicians without extensive US-TFdi training under time constraints in daily practice.

Another advantage resides in improving mechanical ventilation therapy accuracy by evaluating an entire respiratory cycle.

Another advantage resides in using a model-based evaluation of a diaphragm of multiple locations along the diaphragm.

Another advantage resides in relating diaphragm thickness and strain by speckle tracking.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative mechanical ventilation system in accordance with the present disclosure.

FIG. 2 shows an example flow chart of operations suitably performed by the system of FIG. 1.

FIGS. 3-5 show data sets comprising diaphragm thickness and strain generated by the system of FIG. 1.

### DETAILED DESCRIPTION

As used herein, the singular form of “a,” “an,” and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

One approach for improving reproducibility and accuracy of daily (or other frequent) TFdi measurements might be improved clinical procedures such as enforcing a requirement that the same clinician perform the TFdi measurement each day, marking the patient with a fiducial mark indicating where the ultrasound probe should be placed, or so forth. However, the TFdi measurement is typically performed using a handheld ultrasound probe, and hence the positioning of this probe (e.g., location, angulation, force held against the torso, and so forth) respective to the diaphragm can be expected to vary from one measurement to the next even if such improved clinical procedures are implemented.

In embodiments disclosed herein, a different approach is used. In the academic field of Computer and Robot Vision, robust techniques have been developed to reconstruct simultaneously the 3D positions and attitudes of a monocular camera, and the observed unknown 3D scene Simultaneous Localization and Mapping (SLAM) (see, e.g., A. J. Davison, I. D. Reid, N. D. Molton, and O. Stasse, “MonoSLAM: Real-Time Single Camera SLAM,” IEEE Transactions on Pattern Analysis and Machine Intelligence, vol. 29, no. 6, pp. 1052-1067, 2007; Zaffar, Mubariz, Shoaib Ehsan, R. Stolkin and Klaus Dieter Mcdonald-Maier. “Sensors, SLAM and Long-term Autonomy: A Review.” 2018 NASA/ESA Conference on Adaptive Hardware and Systems (AHS) (2018): 285-290]. These types of techniques are repurposed here to calculate TFdi or another diaphragm thickness metric for a given session based on received ultrasound imaging data of the diaphragm of the patient referenced to a reference frame using a patient-specific registration model, which may for example employ a SLAM methodology. In one approach, the patient-specific registration model is constructed on the basis of calibration ultrasound imaging data of the diaphragm of the specific patient acquired during inspiration and expiration while the patient undergoes mechanical ventilation therapy, and with the handheld ultrasound transducer positioned at different positions respective to the diaphragm of the patient, and with calibration respiratory cycle data also obtained over the calibration period from the mechanical ventilator and/or respiratory sensors. Thus, rather than trying to force the clinician to reproducibly place the handheld ultrasound probe in exactly the same position for each TFdi measurement, the measurement is instead mapped to the reference frame, thereby providing improved reproducibility and accuracy. The more accurate and reproducible TFdi measurements can be used for various purposes, such as assessing patient work-of-breath (WoB), determining an optimal time and/or procedure for weaning the patient off the mechanical ventilator, and/or so forth.

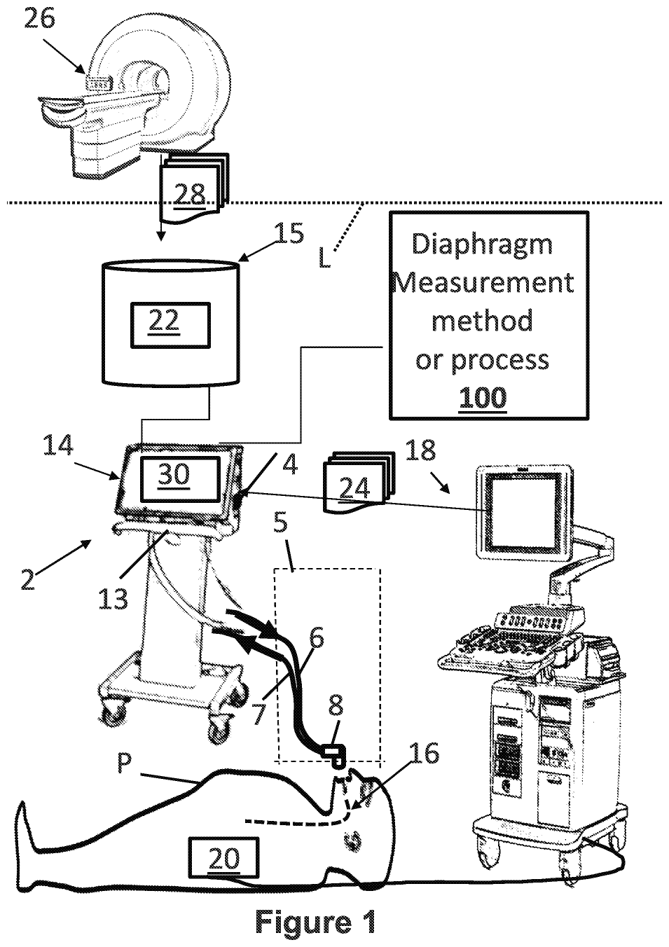

With reference to FIG. 1, a diaphragm measurement device 1 is shown. A mechanical ventilator 2 is configured to provide ventilation therapy to an associated patient P is shown. As shown in FIG. 1, the mechanical ventilator 2 includes an outlet 4 connectable with a patient breathing circuit 5 to delivery mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT) 16, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes at least one electronic processor or controller 13 (e.g., an electronic processor or a microprocessor), a display device 14, and a non-transitory computer readable medium 15 storing instructions executable by the electronic controller 13.

FIG. 1 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 also shows an ultrasound (US) medical imaging device 18 that is used to measure the TFdi or other diaphragm thickness metric. As described herein, the ultrasound medical imaging device 18 is used to acquire ultrasound images or measurement of the patient P. The illustrative embodiments employ brightness mode (B-mode) ultrasound imaging to assess the diaphragm thickness metric. However, other types of ultrasound imaging or data are contemplated, such as motion mode (M-mode) data collected as a single ultrasound line over a time interval, or so forth.

In a more particular example, the medical imaging device 18 includes an ultrasound transducer 20 that is handheld or wearable by the patient P (e.g., on the abdomen or chest of the patient P in position to image the diaphragm of the patient, as shown in FIG. 1). The US transducer 20 is positioned to acquire US imaging data (i.e., US images) 24 of the diaphragm of the patient P. For example, the US transducer 20 is configured to acquire imaging data of a diaphragm of the patient P, and more particularly US imaging data related to a thickness of the diaphragm of a patient P during inspiration and expiration while the patient P undergoes mechanical ventilation therapy with the mechanical ventilator 2. The electronic processor 13 controls the ultrasound imaging device 18 to receive the ultrasound imaging data 24 of the diaphragm of the patient P from the handheld US transducer 20.

In the case of a handheld probe used to acquire diaphragm thickness metric values over multiple sessions, e.g. once a day, the clinician operating the ultrasound device 18 will typically attempt to hold the ultrasound probe 20 positioned in the same way respective to the diaphragm of the patient for each measurement. However, in practice there is expected to be some variation in the position and/or angulation of the ultrasound probe 20 and/or the pressure used to hold it against the patient P from day to day (or more generally from one measurement to the next). It may be that different clinicians perform successive measurements due to varying work shifts and other considerations. Even if the same clinician performs successive measurements some variation in placement of the ultrasound probe 20 is to be expected. A further complication in reproducible probe placement is that the target diaphragm is not directly visible so that the clinician must estimate its position within the torso based on his or her anatomical knowledge. In cases where the ultrasound probe 20 is a wearable probe, variation in probe position can be expected due to patient movement (either volitional due to the patient or movement of an incapacitated patient by medical personnel for hygienic reasons or so forth), slippage of the fastening harness or mechanism used to hold the ultrasound probe 20, and/or so forth. Furthermore, the clinician is expected to identify the end-expiration and end-inspiration times in order to accurately compute the TFdi or other diaphragm thickness metric, and this may be difficult and can introduce further degradation in reproducibility.

To compensate for such measurement-to-measurement variations, the non-transitory computer readable medium 15 stores a patient-specific registration model 22 for referencing the ultrasound imaging data 24 (acquired by the medical imaging device 18) to a reference frame. The patient-specific registration model 22 can be represented by various mathematical approaches: e.g., as an explicit biophysical model (e.g., organ surface triangle meshes as a function of respiratory phase), or as an implicit ML model (e.g., a deeply layered convolutional or recursive artificial neural network encoder, CNN, RNN, or decision trees, Random Forest, gradient boosting machine), or as a high-dimensional non-linear embedding.

An optional additional imaging device (e.g., a CT imaging device 26 as shown in FIG. 1) may acquire one or more CT images 28 of the patient P, and this may optionally serve as further input for constructing the patient-specific registration model 22. For example, if the model 22 is an explicit biophysical model, then the CT images 28 can be used to construct a patient-specific anatomical model of the specific patient P. It should be noted that the CT imaging device 26 may not be located in the same room, or even the same department, as the mechanical ventilator 2. For example, the CT imaging device 26 may be located in a radiology laboratory while the mechanical ventilator 2 may be located in an intensive care unit (ICU), cardiac care unit (CCU), in a hospital room assigned to the patient P, or so forth. This is diagrammatically indicated in FIG. 1 by separator line L.

The non-transitory computer readable medium 15 stores instructions executable by the electronic controller 13 to perform a diaphragm measurement method or process 100.

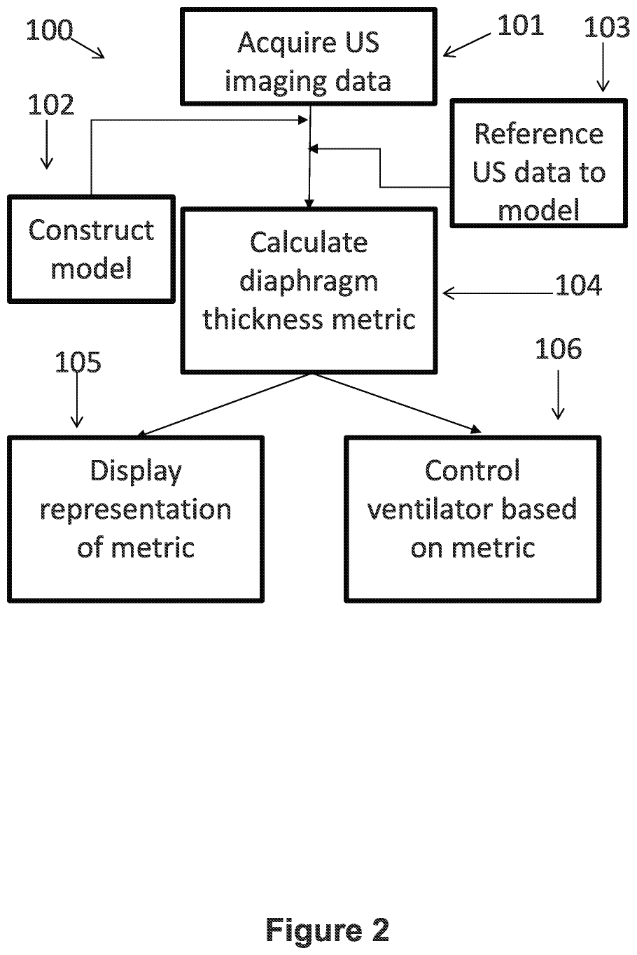

With reference to FIG. 2, and with continuing reference to FIG. 1, an illustrative embodiment of the diaphragm measurement method 100 is diagrammatically shown as a flowchart. At an operation 101, the US imaging data 24 of the diaphragm of the patient P is received during inspiration and expiration while the patient undergoes mechanical ventilation therapy with the mechanical ventilator 2.

At an operation 102, the patient-specific registration model 22 is constructed in an initialization phase of the diaphragm measurement device 1. To do so, in one embodiment, calibration US imaging data 24 of the diaphragm of the patient during inspiration and expiration while the patient P undergoes mechanical ventilation therapy with the mechanical ventilator 2 is received by the electronic controller 13 over a calibration time period. The calibration ultrasound imaging data 24 are acquired with the handheld ultrasound transducer 20 positioned at a plurality of different positions respective to the diaphragm of the patient P. In one contemplated approach, the system may prompt the user to change the position of the handheld ultrasound probe 20, and this prompting may be repeated to provide data for (without loss of generality) N different positions of the ultrasound probe (where N is an integer greater than or equal to two, and in some embodiments more preferably a larger number such as N=4 or N=5). These different probe positions may include different probe angulation positions as well, and/or differences in the amount of pressure used to press the probe 20 against the torso of the patient P. At each probe position, US data are preferably acquired for at least one full respiratory cycle including at least one end-expiration point and at least one end-inspiration point. The N thusly sampled probe positions preferably cover the range of probe positions that may credibly be expected to occur across day-to-day TFdi measurements. This provides ample data for subsequent registering of ultrasound imaging data of the diaphragm of the patient to the reference frame using the patient-specific registration model. However, some extrapolation to unsampled probe positions is readily achievable using SLAM or other spatial registration techniques or the like. Calibration respiratory cycle data tracking respiration of the patient during the calibration time period is also received by the electronic controller 13. The respiratory cycle data can be used to determine when the prompts to change probe position are issued, and are also used to accurately identify the end-expiration and end-inspiration points correlated in time with the (time-stamped) US data. The patient-specific registration model 22 is then constructed based on the calibration ultrasound imaging data and the calibration respiratory cycle data.

In another embodiment of the model construction operation 102, the CT imaging device 26 is used to acquire one or more CT image(s) 28 of a torso of the patient P, which are received by the electronic controller 13. These images are not necessarily acquired while the patient P is on mechanical ventilation, but instead may be acquired (for example) prior to intubation of the patient. Furthermore, other medical imaging modalities such as magnetic resonance imaging (Mill) that provide anatomical information can be used to provide the images 28 as Mill images or so forth. The patient-specific registration model 22 is then constructed including the diaphragm and surrounding organs based on the CT image(s) 28. Missing parts of the patient-specific registration model 22 can be filled in using data earlier analyzed patients, using ‘similar’ points in the patient space.

The patient-specific registration model 22 is constructed as an anatomical model of the patient P, or as an artificial neural network (ANN) model or other machine learning (ML) model. With the patient-specific registration model 22 constructed, it can then be used to provide TFdi or other diaphragm thickness metric measurements with improved reproducibility, as described next.

At an operation 103, US imaging data 24 of the diaphragm of the patient P received during a measurement of the diaphragm thickness metric is referenced to the reference frame using the patient-specific registration model 22 in a use phase of the diaphragm measurement device 1. To do so, in one embodiment, the received US imaging data 24 is acquired by holding an ultrasound probe 20 respective to the diaphragm of the patient P to acquire the US imaging data 24. The US imaging data 24 is then spatially registered to the reference frame comprising a reference orientation of the ultrasound probe 20 respective to the diaphragm of the patient P. In another embodiment, the received US imaging data 24 is spatially registered to the reference frame comprising reference ultrasound probe 20 orientation (e.g., location, attitude, and patient anatomy) respective to the diaphragm of the patient P using the patient-specific registration model 22. The spatially registration process can comprise, for example, a simultaneous localization and mapping (SLAM) process.

At an operation 104, a diaphragm thickness metric can be calculated based on the received US imaging data 24 of the diaphragm of the patient P referenced to the reference frame using the patient-specific registration model 22. In one example, the diaphragm thickness metric includes a diaphragm thickening ratio indicative of a diaphragm thickness during inspiration relative to a diaphragm thickness during expiration. In another example, the diaphragm thickness metric includes a mean diaphragm thickness over multiple respiratory cycles.

Advantageously, using the patient-specific registration model 22, the electronic controller 13 compensates for the unknown variations in probe location, attitude (angulation), pressure etc. This allows for greater flexibility in clinician positioning of the ultrasound probe 20, as day-to-day variations in such position can be compensated using the patient-specific registration model 22. The compensation is achieved in the way that the patient-specific registration model 22 is varied (i.e., adapted) until the newly incoming US imaging data 24 (including images and strain measurements) is optimally predicted or reproduced for a probable probe location and attitude at one or a series of respiratory phase points. Residual changes in the patient-specific registration model 22, which cannot be predicted or reproduced by changes in probe location/attitude, are attributed changes in anatomy and propagated back to the predicted or reproduced. The adapted patient-specific registration model 22 is then used to provide a compensated diaphragm thickness metric (as opposed to the error-prone actual diaphragm thickness metric).

The adaptation of the patient-specific registration model 22 including the unknowns (probe location/attitude, anatomical changes, etc.) can be achieved by established techniques such as convex optimization, iterative back propagation, etc.

At an operation 105, a representation 30 of the calculated diaphragm thickness metric is displayed on the display device 14 of the mechanical ventilator 2. In some embodiments, at an operation 106, the mechanical ventilator 2 can be controlled to adjust one or more parameters of the mechanical ventilation therapy delivered to the patient based on the calculated diaphragm thickness metric.

In some embodiments, the diaphragm measurement method 100 can be repeated for successive sessions and to generate a trendline for the calculated diaphragm thickness metric. An indication of an outlier can be displayed on the display device 14 if a repetition of the diaphragm measurement method calculates the diaphragm thickness metric deviating from the trendline by greater than a threshold deviation.

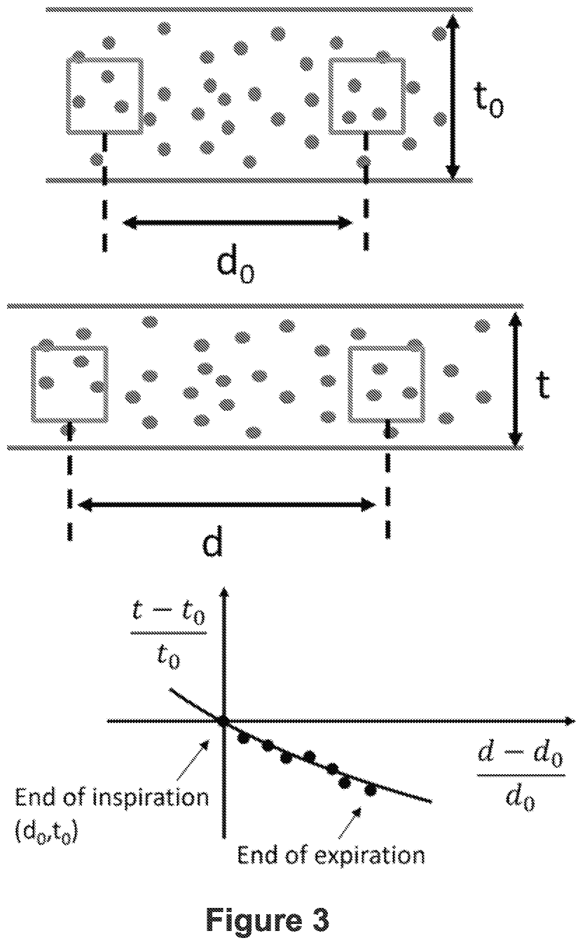

FIG. 3 shows an example of generating the diaphragm thickness metric. A functional relation between diaphragmatic thickness (i.e., distance between diaphragmatic hyperechoic lines) and diaphragmatic strain (as determined by speckle tracking) is established for the various indicated grid locations. The dots shown in the representations represent US speckles.

The top representation represents a diaphragm in a contracted state, and the middle representation represents the diaphragm in a relaxed state. When the diaphragm is elongated, it will get thinner (i.e. t<t0). The distance between the speckles along the length of the diaphragm increases when elongating the diaphragm. Two blocks or squares can be shown to keep track of the speckles inside these blocks. This can be done using computer vision tracking software implemented in the electronic controller 13 that recognizes the unique speckle pattern inside these blocks in each frame (e.g. by block matching). When elongating the diaphragm, the horizontal distance between the squares will increase (i.e. d>d0) since the speckles inside these squares will move outwards in horizontal direction. t0 and d0 is the thickness and distance at a certain starting point (i.e., reference point), for example, at the beginning of the measurement or/and at a defined moment in the breathing cycle. This is at the end of inspiration where the diaphragm thickness has its maximum value and minimum strain value).

The bottom representation in FIG. 3 shows a relation between relative thickness change in a breathing cycle (t-t0)/t0 and relative displacement changes between the squares or strain (d-d0)/d0 is depicted (see, e.g., Sivesgaard, et. Al. “Speckle Tracking Ultrasound is Independent of Insonation Angle and Gain: An In Vitro Investigation of Agreement with Sonomicrometry. American Society of Echocaridoography. Doi: 10.1016/j.echo.2009.04.028). The various dots represent measurement points at various moments in the breathing cycle, i.e. the dots at the left correspond to end of inspiration (fully contracted diaphragm) and dots at the right with end of expiration (fully relaxed, elongated diaphragm). Advantageously, as seen in this plot data are collected across the respiratory cycle, and not only at end-inspiration and end-expiration. This can enable curve fitting or other statistical techniques applied to this rich data set to compute the diaphragm thickness metric more accurately.

In daily usage, only one or few different locations need to be recorded. However, by the field of view (FOV) of the US probe 20, and natural spread of the probe locations, the patient-specific registration model 22 is updated in its spatial extent using certain regularization conditions (continuity, smoothness, elasticity, and so forth). Thus, non-single-location but location-generalizing comprehensive trends of the muscular development trends are reported.

All diaphragmatic changes may be reported with uncertainty estimates and/or confidence intervals, as derived from the model variability as it responds to slightly modified inputs (artificial perturbations), to indicate whether the changes can be considered significant.

Angulation between probe and diaphragm is a dependency for the absolute diaphragm thickness. However, the fractional change during tidal breathing over the respiratory cycle is invariant to angulation provided that the location on the diaphragm has been estimated using the patient-specific registration model 22 and assuming the angulation is constant during the data acquisition across the respiratory cycle.

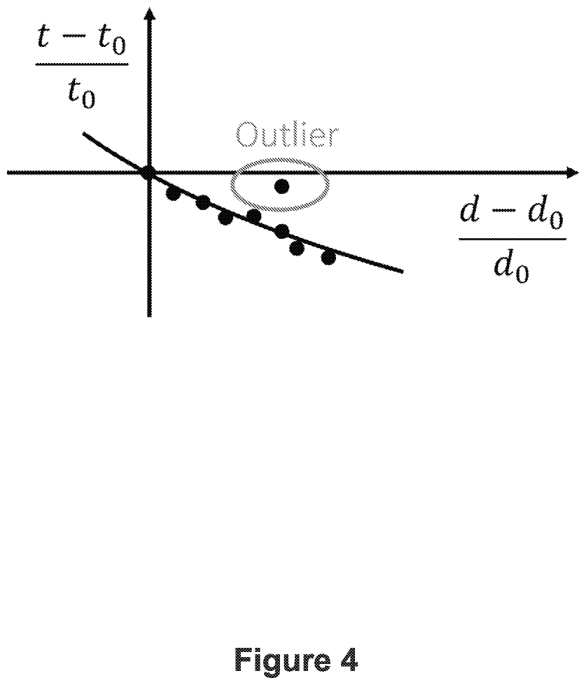

FIG. 4 shows an example detecting outliers in the US imaging data 24. Functional dependencies of diaphragmatic thickness and strain are determined and compared with the pre-determined functional relation. Based on this comparison outliers are recognized and removed, e.g., when deviating >10% from the pre-determined functional relation. This outlier removal can further improve accuracy of the diaphragm thickness metric measurements. The diaphragm measurement device 1 in one approach indicates to the user if/when sufficient data for the (daily) model adaptation has been collected, or further measurement time is required to dissolve ambiguities. The internal model may optionally be continuously personalized/specified, with more data points becoming available for model fitting. Probabilistic techniques can be employed to weigh between short-term adaptations (refinements) to be compensated versus long term change trends to be reported. The course of the patient development over the treatment period is computed and reported as trend curves.

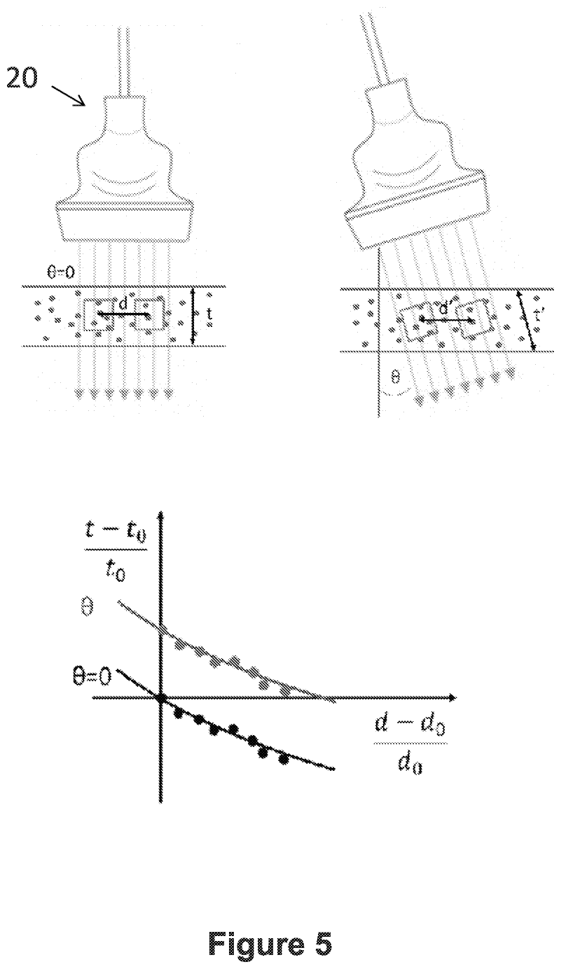

FIG. 5 shows a representation of how the US imaging device 18 is used to obtain the US imaging data 24 and used to determine the diaphragm thickness metric as shown in FIG. 3. In case an operator-caused change in angulation has occurred during the respiratory cycle, then this may be recognized by virtue of superficial anatomical structures not moving as a function of the respiratory cycle, e.g., ribs. The amount of angulation change can be estimated by the SLAM process and be corrected. Changing the angle between the probe 20 and the diaphragm will lead to an apparent change in diaphragm thickness. However, speckle tracking derived diaphragmatic strain is probe angle independent. Hence, the functional relation between thickness and strain will shift along the relative thickness axis depending on the angle between the probe 20 and the diaphragm. After calibration in the initialization phase this effect can be used to detect a change in angulation. Also, the patient-specific registration model 22 can be updated on the probe angle. The angle can be calculated, and thickness measurements can be compensated for the angle.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A diaphragm measurement device, comprising:
a non-transitory storage medium storing a patient-specific registration model for referencing ultrasound imaging data to a reference frame; and
at least one electronic processor programmed to perform a diaphragm measurement method including:
receiving ultrasound imaging data of a diaphragm of a patient during inspiration and expiration while the patient undergoes mechanical ventilation therapy with a mechanical ventilator;
calculating a diaphragm thickness metric based on the received ultrasound imaging data of the diaphragm of the patient referenced to the reference frame using the patient-specific registration model; and
displaying, on a display device, a representation of the calculated diaphragm thickness metric.

2. The device of claim 1, wherein the diaphragm thickness metric includes a diaphragm thickening ratio indicative of a diaphragm thickness during inspiration relative to a diaphragm thickness during expiration.

3. The device of claim 1, wherein the diaphragm thickness metric includes a mean diaphragm thickness over multiple respiratory cycles.

4. The device of claim 1, further comprising:
an ultrasound imaging device including a handheld ultrasound transducer, wherein the at least one electronic processor controls the ultrasound imaging device to receive the ultrasound imaging data of the diaphragm of the patient from the handheld ultrasound transducer.

5. The device of claim 4, wherein the at least one electronic processor is further programmed to:
over a calibration time period, receive calibration ultrasound imaging data of the diaphragm of the patient during inspiration and expiration while the patient undergoes mechanical ventilation therapy with the mechanical ventilator, wherein the calibration ultrasound imaging data are acquired with the handheld ultrasound transducer positioned at a plurality of different positions respective to the diaphragm of the patient;
receive calibration respiratory cycle data tracking respiration of the patient during the calibration time period; and
construct the patient-specific registration model based on the calibration ultrasound imaging data and the calibration respiratory cycle data.

6. The device of claim 1, wherein the at least one electronic processor is further programmed to:
receive a computed tomography (CT) image of a torso of the patient; and
construct the patient-specific registration model as an anatomical model of the patient including the diaphragm and surrounding organs based on the CT image.

7. The device of claim 6, wherein the patient-specific registration model is constructed as an anatomical model or as an artificial neural network (ANN) model.

8. The device of claim 1, wherein the referencing of the received ultrasound imaging data of the diaphragm of the patient to the reference frame using the patient-specific registration model includes:
spatially registering the received ultrasound imaging data to the reference frame comprising a reference orientation of an ultrasound probe respective to the diaphragm.

9. The device of claim 1, wherein the referencing of the received ultrasound imaging data of the diaphragm of the patient to the reference frame using the patient-specific registration model includes:
spatially registering the received ultrasound imaging data to the reference frame comprising a reference ultrasound probe orientation respective to the diaphragm of the patient using the patient-specific registration model.

10. The device of claim 9, wherein the patient-specific registration model is configured to apply a simultaneous localization and mapping (SLAM) process to spatially register the received ultrasound imaging data to the reference ultrasound probe orientation.

11. The device of claim 1, wherein the at least one electronic processor is further programmed to:
repeat the diaphragm measurement method for successive sessions and to generate a trendline for the calculated diaphragm thickness metric; and
display, in the display, an indication of an outlier if a repetition of the diaphragm measurement method calculates the diaphragm thickness metric deviating from the trendline by greater than a threshold deviation.

12. The device of claim 1, wherein the at least one electronic controller is configured to:
control a mechanical ventilator to adjust one or more parameters of the mechanical ventilation therapy delivered to the patient based on the calculated diaphragm thickness metric.

13. The device of claim 1, further comprising:
a mechanical ventilator configured to deliver mechanical ventilation therapy to the patient.

14. A diaphragm measurement method comprising, with at least one electronic controller:
receiving ultrasound imaging data of a diaphragm of a patient during inspiration and expiration while the patient undergoes mechanical ventilation therapy with a mechanical ventilator;
calculating a diaphragm thickness metric based on the received ultrasound imaging data of the diaphragm of the patient referenced to a reference frame using a patient-specific registration model; and
displaying, on a display device, a representation of the calculated diaphragm thickness metric.

15. The device of claim 14, further including:
repeating the diaphragm measurement method for successive sessions and to generate a trendline for the calculated diaphragm thickness metric; and
displaying, in the device, an indication of an outlier if a repetition of the diaphragm measurement method calculates the diaphragm thickness metric deviating from the trendline by greater than a threshold deviation.
